(19) United States
(12) Patent Application Publication
(10) Pub. No.: US 2024/0245854 A1
(43) Pub. Date: Jul. 25, 2024

(54) PUSHING DEVICE FOR DRUG DELIVERY DEVICE

(71) Applicants: TOBIOS INC, Seoul (KR); Seoul National University R&DB Foundation, Seoul (KR)

(72) Inventors: Young Bin CHOY, Seoul (KR); BEOM KANG HUH, Seoul (KR); Song Ah KO, Seoul (KR)

(21) Appl. No.: 18/418,038

(22) Filed: Jan. 19, 2024

(30) Foreign Application Priority Data
Jan. 20, 2023 (KR) 10-2023-0008948

(51) Int. Cl. A61M 5/142

(52) U.S. Cl. CPC A61M 5/1428; A61M 2205/3327

(57) ABSTRACT

Provided is a pushing device for a drug delivery device that is implanted in the body and coupled to a drug delivery device, which includes a press button for discharging a drug stored inside a housing to the outside, to selectively press the press button, the pushing device including: a body portion detachably coupled to the drug delivery device with skin tissue placed therebetween; and a presser supported to be movable from the body portion in a longitudinal direction thereof and disposed to face the press button.

### CROSS-REFERENCE TO RELATED APPLICATION

This application claims priority to and the benefit of Korean Patent Application No. 10-2023-0008948, filed on Jan. 20, 2023, the disclosure of which is incorporated herein by reference in its entirety.

### BACKGROUND

### Technical Field

The present disclosure relates to a pushing device for a drug delivery device that can be coupled to a drug delivery device implanted in the body, and more particularly, to a pushing device for a drug delivery device that can be coupled to a drug delivery device with skin tissue placed therebetween to selectively press a button provided on the drug delivery device.

### Discussion of Related Art

Diabetes is known as a common disease that about two hundred million people around the world currently have, and complications relating to diabetes involve human, social, and economic loss and thus are considered an important health problem.

Diabetes is a disease that develops when the human body fails to produce sufficient insulin or fails to properly utilize insulin and thus glucose in blood (blood glucose) is not converted into energy. Therefore, patients with diabetes need to continuously measure blood glucose or be administered with insulin as necessary.

In recent years, a drug delivery device allowing a patient to self-inject insulin has been developed and used. With such a drug delivery device, insulin is directly administered through a blood vessel or subcutaneous fat by the user, and thus in order to constantly maintain a certain insulin level in the body, it is important to inject a fixed amount of insulin at a fixed time.

However, a prescribed fixed amount of insulin is often not injected due to a mistake of a user using the drug delivery device, and in some cases, a user forgets whether he or she has injected insulin and injects insulin more than a predetermined number of times or does not inject insulin at all.

### SUMMARY

The present disclosure is directed to a pushing device for a drug delivery device, which includes a press button for discharging a drug stored inside a housing, wherein the device is configured to count the number of times the press button has been pressed to prevent overuse or non-use of the drug delivery device.

According to an aspect of the present disclosure, there is provided a pushing device for a drug delivery device that can be implanted in the body and coupled to a drug delivery device, which includes a press button for discharging a drug stored inside a housing, the pushing device comprising: a body portion detachably coupled to the drug delivery device configured such that skin tissue can be placed therebetween; and a presser supported to be movable from the body portion in a longitudinal direction thereof and disposed to face the press button.

Also, the body portion may have an insertion groove portion formed to be recessed in one side to allow the drug delivery device to be inserted thereinto and a through-hole formed in an upper portion in a vertical direction to allow the presser to be coupled.

Also, the presser may have a support groove portion formed along a circumference by one portion of a side portion of the presser being recessed and an upper step and a lower step formed at an upper portion and a lower portion, respectively, and one portion of the upper step may be disposed in the through-hole.

Also, the body portion may include a protruding member protruding toward a lower inner side of the through-hole, and the lower step of the presser may be disposed under the protruding member and interfere with the protruding member.

Also, the presser may be elastically supported on the body portion by a spring member, and the spring member may be disposed between the upper step of the presser and the protruding member of the body portion.

Also, a magnetic body may be provided on a portion of the presser, for example, a lower portion, and an electromagnet configured to selectively form an attractive force relative to the magnetic body according to a current applied from the outside may be provided on the insertion groove portion.

Also, at least one guide protrusion longitudinally formed in the vertical direction may be provided along an outer circumference of the presser, and a guide groove portion configured to have the guide protrusion inserted thereinto and guide movement of the presser may be formed in an inner wall of the body portion in which the through-hole is formed.

Also, a magnetic body may be installed on a contact portion between the presser and the press button to prevent sliding of the presser.

Also, the pushing device may further include: a measurer configured to count a number of times an external force is applied to the presser and the presser presses the press button; and a display configured to output the number of times the press button is pressed that is counted by the measurer.

Also, the measurer may measure click information of the press button provided on the drug delivery device to count the number of times the press button is pressed.

The pushing device may further include: a rotary gear portion mounted on the body portion and configured to generate a rotational force; and an engaging portion attached to the support groove portion and engaged with the rotary gear portion to allow the presser to move in the longitudinal direction as the rotary gear portion rotates.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a front view of a pushing device for a drug delivery device according to one embodiment of the present disclosure;

FIG. 2 is a lateral cross-sectional view of the pushing device for a drug delivery device illustrated in FIG. 1;

FIG. 3 is a view illustrating a state in which the pushing device for a drug delivery device of FIG. 2 is coupled to a drug delivery device;

FIG. 4 is a view illustrating a state in which the pushing device for a drug delivery device of FIG. 3 presses a press button of the drug delivery device;

FIG. 5 is a view illustrating a state in which, while the drug delivery device is implanted in skin tissue, the pushing device of FIG. 2 is coupled to the drug delivery device;

FIG. 6 is a cross-sectional view of a pushing device for a drug delivery device according to another embodiment of the present disclosure;

FIG. 7 is a cross-sectional view of a pushing device for a drug delivery device according to still another embodiment of the present disclosure;

FIG. 8 is a cross-sectional view of a pushing device for a drug delivery device according to yet another embodiment of the present disclosure;

FIG. 9 is a cross-sectional view of a pushing device for a drug delivery device according to yet another embodiment of the present disclosure; and

FIG. 10 is a cross-sectional view of a pushing device for a drug delivery device according to yet another embodiment of the present disclosure.

### DETAILED DESCRIPTION

Hereinafter, exemplary embodiments of a pushing device for a drug delivery device will be described in detail with reference to the accompanying drawings. Here, like components will be denoted by like reference numerals, and repeated description and detailed description of known functions and configurations that may unnecessarily obscure the gist of the disclosure will be omitted. Embodiments of the disclosure are only provided to more completely describe the disclosure to those of ordinary skill in the art. Therefore, shapes, sizes, and the like of components illustrated in the drawings may have been exaggerated for clearer description.

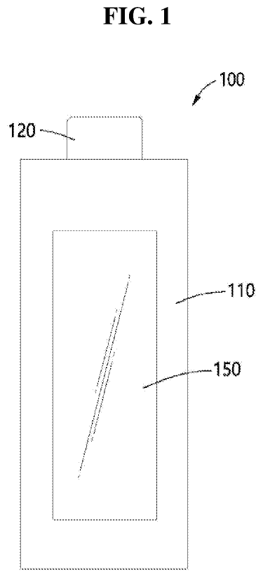

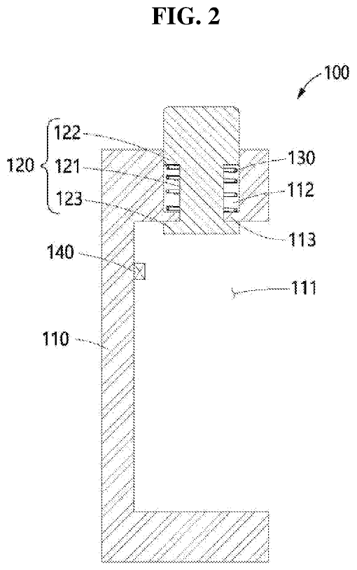

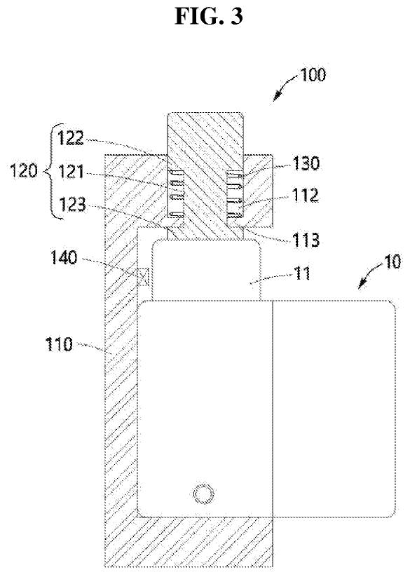

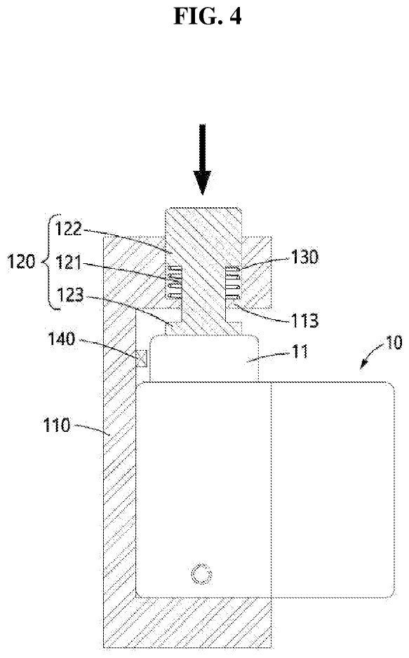

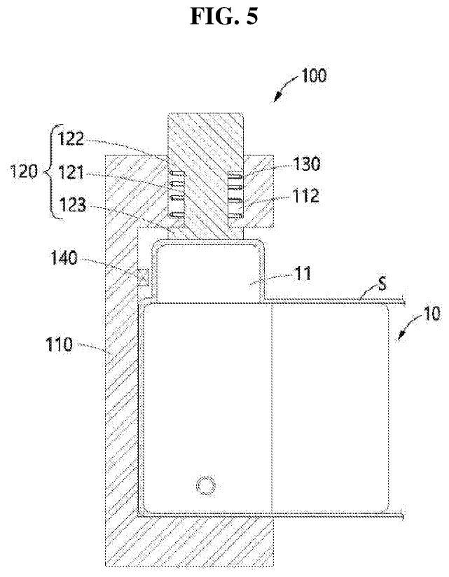

FIG. 1 is a front view of a pushing device for a drug delivery device according to one embodiment of the present disclosure, FIG. 2 is a lateral cross-sectional view of the pushing device for a drug delivery device illustrated in FIG. 1, FIG. 3 is a view illustrating a state in which the pushing device for a drug delivery device of FIG. 2 is coupled to a drug delivery device, FIG. 4 is a view illustrating a state in which the pushing device for a drug delivery device of FIG. 3 presses a press button of the drug delivery device, and FIG. 5 is a view illustrating a state in which, while the drug delivery device is implanted in skin tissue, the pushing device of FIG. 2 is coupled to the drug delivery device.

Referring to FIGS. 1 to 5, a pushing device 100 for a drug delivery device may include a body portion 110 and a presser 120.

The pushing device 100 for a drug delivery device according to the present embodiment is implanted in the body and coupled to a drug delivery device 10, which includes a press button 11 for discharging a drug stored inside a housing to the outside, to selectively press the press button 11. The pushing device 100 for a drug delivery device may be compatibly used with a drug delivery device described in Korean Patent Application Publication No. 10-2021-0096382. However, the compatible drug delivery device 10 is not limited thereto, and any other drug delivery device 10 may be compatibly used as long as the drug delivery device 10 has the press button 11 provided on an outer side thereof.

Meanwhile, the drug delivery device 10 may be implanted in tissue of skin S as illustrated in FIG. 5, and during use, the pushing device 100 according to the present disclosure may be coupled to the drug delivery device 10 implanted in the tissue of the skin S, with the skin placed between the pushing device 100 and the drug delivery device 10.

The body portion 110 may be detachably coupled to the drug delivery device 10 with the tissue of the skin placed therebetween. For example, the body portion 110 may have an insertion groove portion 111 formed to be recessed in one side to allow the drug delivery device 10 to be inserted thereinto and a through-hole 112 formed in an upper portion in a longitudinal direction to allow the presser 120, which will be described below, to be inserted thereinto and coupled. Here, the longitudinal direction may be a direction identical to a direction in which the press button 11 moves, that is, a direction in which the drug is injected.

The insertion groove portion 111 formed in the body portion 110 may be formed to match an outer shape of the drug delivery device 10 so that, when the drug delivery device 10 and the insertion groove portion 111 are coupled to each other, a gap is not formed therebetween. In particular, in a case where the insertion groove portion 111 and the drug delivery device 10 can each have an exterior formed in the shape of a quadrangular column (although other shapes are possible), when the drug delivery device 10 is inserted into the insertion groove portion 111, the drug delivery device 10 can be prevented from rotating from the insertion groove portion 111 to allow more stable injection of the drug.

The presser 120 may be supported to be movable from the body portion 110 in the longitudinal direction and may be disposed to face the press button 11. For example, in a case where the press button 11 is positioned at an upper side when the drug delivery device 10 is inserted into the insertion groove portion 111, the presser 120 may be disposed above the press button 11 and positioned to face the press button 11. Accordingly, when a user applies a downward external force to the presser 120, the presser 120 comes in contact with the press button 11 of the drug delivery device 10, and the presser 120 and the press button 11 may simultaneously move downward.

The presser 120 may have a support groove portion 121 formed along a circumference by one portion of a side portion of the presser 120 being recessed and an upper step 122 and a lower step 123 formed at an upper portion and a lower portion, respectively, and one portion of the upper step 122 may be disposed in the through-hole 112. Due to such a structure, the presser 120 may move in the vertical direction, for example, the longitudinal direction of the body portion 110, in the through-hole 112 of the body portion 110.

Specifically, the body portion 110 may include a protruding member 113 protruding toward a lower inner side of the through-hole 112, and the lower step 123 of the presser 120 may be disposed at a lower portion of the protruding member 113 and interfere with the protruding member 113. In this way, since the lower step 123 of the presser 120 interferes with the protruding member 113 of the body portion 110, the presser 120 may be caught in the body portion 110 when being restored to an upper portion after moving downward, and thus the presser 120 may be prevented from falling out of the through-hole 112 of the body portion 110.

Meanwhile, the presser 120 may be supported to be movable from the body portion 110 by compressive and restoring forces of a spring member 130. For example, the spring member 130 may be disposed between the upper step 122 of the presser 120 and the protruding member 113 of the body portion 110. Accordingly, the presser 120 may be elastically supported on the body portion 110 by the spring member 130, and thus, when the user applies an external force and presses the presser 120 downward, the presser 120 moves downward due to the compressive force of the spring member 130, and when the user releases the external force, the presser 120 moves upward and returns to its original position due to the restoring force of the spring member 130.

In the present embodiment, the presser 120 has been described as moving inside the body portion 110 by the spring member 130, but the presser 120 may also be restored by a power source such as an actuator. In addition, the presser 120 may also move due to a restoring force of the drug delivery device 10 itself without the spring member 130.

According to the present disclosure, the pushing device 100 for a drug delivery device may further include a measurer 140 and a display 150.

The measurer 140 may count the number of times an external force is applied to the presser 120 and the presser 120 presses the press button 11. For example, the measurer 140 may measure click information of the press button 11 provided on the drug delivery device 10 to count the number of times the press button 11 is pressed. Specifically, when the press button 11 provided on the drug delivery device 10 moves due to a force of a certain magnitude applied thereto, the press button 11 is clicked at a certain position such that the force is reduced, and the measurer 140 may count the number of clicks to measure the number of times the press button 11 is pressed. The amount of injected drug may be checked by counting the number of clicks.

In this way, since the pushing device 100 for a drug delivery device is formed to be coupled to the drug delivery device 10 and count the number of times the press button 11 is pressed, overuse of the drug delivery device 10 may be prevented. That is, in a case where a user needs to inject a drug once a day, the user may forget that he or she has injected the drug in the morning and re-inject the drug in the afternoon or may totally forget to inject the drug and not inject the drug at all, and since the number of times the press button 11 is pressed can be counted by the pushing device 100 for a drug delivery device, overuse or non-use of the drug delivery device 10 can be prevented.

Also, since the measurer 140 is configured to count the number of times the press button 11 is pressed, by calculating the amount of injected drug according to the number of clicks, whether a prescribed fixed amount of the drug has been injected can be checked. That is, in a case where 1 U of the drug is released per click, when the number of clicks counted by the measurer 140 is three, it may be calculated that 3 U of the drug has been released from the drug delivery device 10. The amount of injected drug may be output through the display 150 which will be described below.

The measurer 140 may further include a speaker or a light emitting diode (LED) lamp and generate sound or light when counting the number of times the press button 11 is pressed, and thus whether the press button 11 has been pressed normally may be checked aurally and visually, and convenience in use may be further improved. In this way, since whether the press button 11 has been pressed normally can be determined through the measurer 140, incomplete administration can be prevented, and a predetermined amount of drug can be more accurately injected.

The display 150 may output the number of times the press button 11 is pressed that is counted by the measurer 140. For example, the display 150 may be provided on an exterior of the body portion 110 and output the number of times the press button 11 is pressed that is counted by the measurer 140. Here, the pushing device 100 for a drug delivery device may be formed to interwork with a smartphone application or another external device, and in this case, the display 150 may be provided on the external device, instead of the body portion 110, and output the number of times the press button 11 is pressed that is counted by the measurer 140. Alternatively, the display 150 may be provided on both the body portion 110 and the external device.

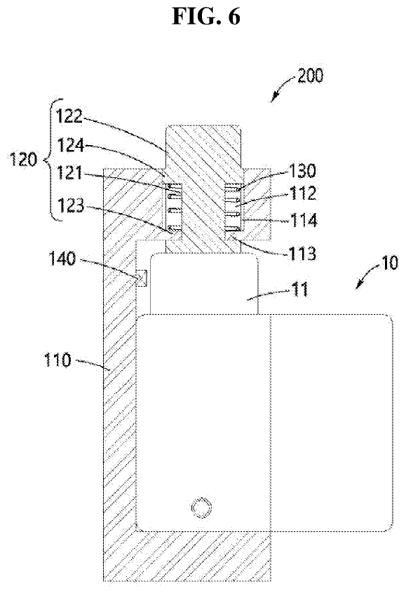

FIG. 6 is a cross-sectional view of a pushing device for a drug delivery device according to another embodiment of the present disclosure.

Referring to FIG. 6, a pushing device 200 for a drug delivery device may further include a guide protrusion 124 formed along an outer circumference of the presser 120 and a guide groove portion 114 formed in an inner wall of the body portion 110. For example, at least one guide protrusion 124 longitudinally formed in the vertical direction may be provided along the outer circumference of the presser 120, and at least one guide groove portion 114 configured to have the guide protrusion 124 inserted thereinto and guide movement of the presser 120 may be provided in the inner wall of the body portion 110 in which the through-hole 112 is formed. Here, the guide protrusion 124 may be formed to protrude in a semispherical shape, and the guide groove portion 114 may also be formed in a shape that corresponds to the guide protrusion 124. Also, the number of guide protrusions 124 and the number of guide groove portions 114 may be the same, and the guide protrusions 124 and the guide groove portions 114 may be provided at positions corresponding to each other.

In this way, since movement of the presser 120 inside the body portion 110 is guided by the guide protrusion 124 and the guide groove portion 114, the presser 120 may be prevented from idly rotating in the body portion 110 when moving downward, and convenience in use may be improved.

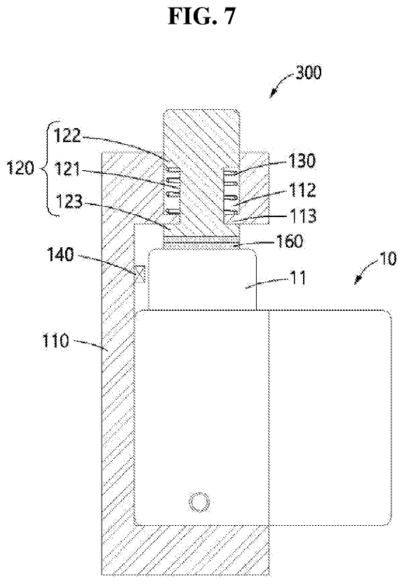

FIG. 7 is a cross-sectional view of a pushing device for a drug delivery device according to still another embodiment of the present disclosure.

Referring to FIG. 7, a pushing device 300 for a drug delivery device may further include a magnetic body 160. For example, the magnetic body 160 may be installed on a contact portion between the presser 120 and the press button 11 to prevent sliding of the presser 120.

Specifically, the magnetic body 160 may be installed on a bottom surface of the presser 120, and another magnetic body 160 may be installed on an upper surface of the press button 11 that faces the presser 120 so that the presser 120 and the press button 11 are able to move while attached to each other by a magnetic force, and accordingly, the presser 120 can be prevented from sliding due to an axis thereof tilting when the presser 120 presses the press button 11.

In the present embodiment, the pair of magnetic bodies 160 have been described as being installed on each of the presser 120 and the press button 11, but the press button 11 itself may be formed of the magnetic body 160 or a metal material, and the magnetic body 160 may be installed on the bottom surface of the presser 120 to prevent sliding of the presser 120. In addition, the press button 11 itself may be formed of the magnetic body 160 or a metal material, and the presser 120 itself may be formed of the magnetic body 160 to prevent sliding of the presser 120.

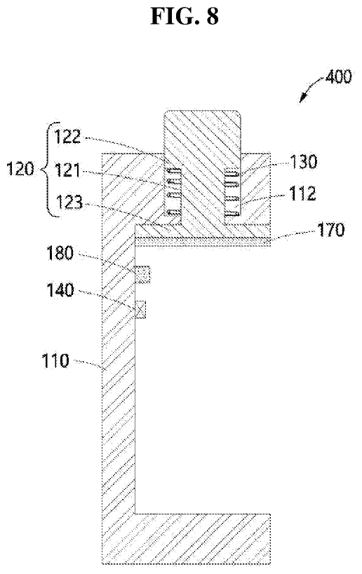

FIG. 8 is a cross-sectional view of a pushing device for a drug delivery device according to yet another embodiment of the present disclosure.

Referring to FIG. 8, the presser 120 of a pushing device 400 for a drug delivery device may be supported to be movable from the body portion 110 by a magnetic force. For example, a magnetic body 170 may be provided on a lower portion of the presser 120, and an electromagnet 180 configured to selectively form an attractive force relative to the magnetic body 170 according to a current applied from the outside may be provided on the insertion groove portion 111. To this end, although not illustrated, the pushing device 400 for a drug delivery device may further include a battery configured to apply a current to the electromagnet 180 and a switch configured to turn the battery on or off.

Thus, when a user turns on the switch, current is applied to the electromagnet 180, and an attractive force relative to the magnetic body 170 is generated, which causes the presser 120 to move downward. When the user turns off the switch, the supply of current applied to the electromagnet 180 is stopped, and the presser 120 moves upward due to the restoring force of the spring member 130.

In addition, although not illustrated, the presser 120 may be moved in the vertical direction by a linear actuator, a hydraulic cylinder, a pneumatic cylinder, or the like. That is, a method of moving the presser 120 in the vertical direction is not limited to that described above and may be implemented in various other ways.

As described above, the pushing device for a drug delivery device may be coupled to the drug delivery device 10 including the press button 11 and count the number of times the press button 11 is pressed, thereby preventing overuse or non-use of the drug delivery device 10.

Also, since whether the press button 11 is clicked normally can be determined through the measurer 140, incomplete administration can be prevented, and a predetermined amount of drug can be more accurately injected.

Also, since the measurer 140 is formed to measure click information of the presser 120, by calculating the amount of injected drug according to the number of clicks, whether a prescribed fixed amount of the drug has been injected can be checked.

Further, since the magnetic body 170 is installed on a contact portion between the presser 120 and the press button 11, the presser 120 can be prevented from sliding due to an axis thereof tilting when the presser 120 presses the press button 11.

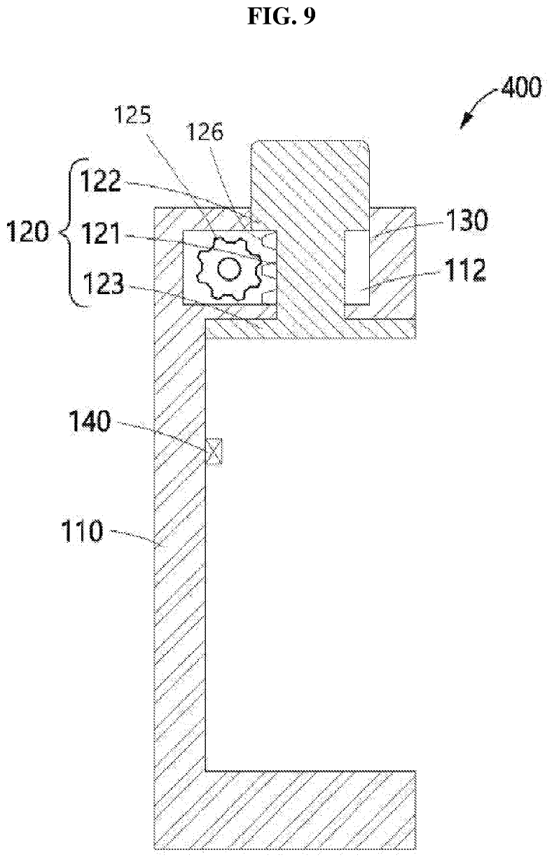

FIG. 9 is a cross-sectional view of a pushing device for a drug delivery device according to yet another embodiment of the present disclosure.

Referring to FIG. 9, a pushing device for a drug delivery device according to yet another embodiment of the present disclosure may further include: a rotary gear portion 125 mounted on the body portion 110 and configured to generate a rotational force; and an engaging portion 126 attached to the support groove portion 121 and engaged with the rotary gear portion 125 to allow the presser 120 to move in the longitudinal direction as the rotary gear portion 125 rotates.

Specifically, the rotary gear portion 125 may be connected to an electric motor and generate a rotational force, or the pushing device may further include a rotary handle portion protruding outward that allows the rotary gear portion 125 to be manually rotated without an electric motor. In a case where an electric motor is included, of course, a small-sized power supply connected to the electric motor may be further included.

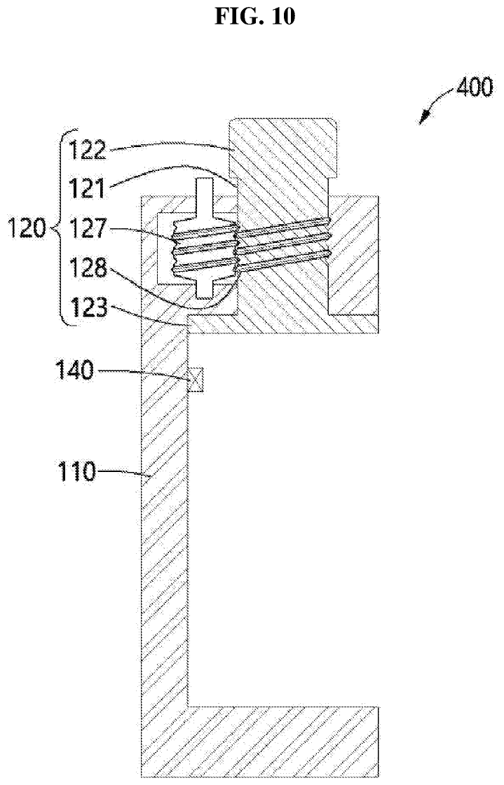

FIG. 10 is a cross-sectional view of a pushing device for a drug delivery device according to yet another embodiment of the present disclosure.

Referring to FIG. 10, a pushing device for a drug delivery device according to yet another embodiment of the present disclosure may further include: a screw portion 127 mounted on the body portion 110 and configured to generate a rotational force; and a screw coupling portion 128 formed in the form of screw threads on the support groove portion 121 and engaged with the screw portion 127 to allow the presser 120 to move in the longitudinal direction as the screw portion 127 rotates.

Specifically, the screw portion 127 may be connected to an electric motor and generate a rotational force, or the pushing device may further include a rotary handle portion protruding outward that allows the screw portion 127 to be manually rotated without an electric motor. In a case where an electric motor is included, of course, a small-sized power supply connected to the electric motor may be further included.

According to the present disclosure, a pushing device for a drug delivery device is coupled to a drug delivery device including a press button and counts the number of times the press button is pressed. Accordingly, it is possible to prevent overuse or non-use of the drug delivery device.

Also, since whether the press button is clicked normally can be determined through a measurer, incomplete administration can be prevented, and a predetermined amount of drug can be more accurately injected.

Also, since the measurer is formed to measure click information of a presser, by calculating the amount of injected drug according to the number of clicks, whether a prescribed fixed amount of the drug has been injected can be checked.

Further, since a magnetic body is installed on a contact portion between the presser and the press button, the presser can be prevented from sliding due to an axis thereof tilting when the presser presses the press button.

The present disclosure has been described above with reference to the embodiments illustrated in the accompanying drawings, but the embodiments are only illustrative, and those of ordinary skill in the art should understand that various modifications and other equivalent embodiments are possible therefrom. Therefore, the true protection scope of the present disclosure should be defined only by the appended claims.

What is claimed is:

1. A pushing device for a drug delivery device configured to be implanted in the body and coupled to a drug delivery device, which includes a press button for discharging a drug stored inside a housing to the outside, to selectively press the press button, the pushing device comprising:
a body portion detachably coupled to the drug delivery device configured such that skin tissue can be placed therebetween; and
a presser supported to be movable from the body portion in a longitudinal direction thereof and disposed to face the press button.

2. The pushing device of claim 1, wherein the body portion comprises an insertion groove portion formed to be recessed in at least one side to allow the drug delivery device to be inserted thereinto and a through-hole formed in an upper portion in the longitudinal direction to allow the presser to be coupled.

3. The pushing device of claim 2, wherein the presser comprises a support groove portion formed along a circumference by a side portion of the presser being recessed and an upper step and a lower step formed at an upper portion and a lower portion, and one portion of the upper step is disposed in the through-hole.

4. The pushing device of claim 3, wherein the body portion comprises a protruding member protruding toward a lower inner side of the through-hole, and the lower step of the presser is disposed under the protruding member and interferes with the protruding member.

5. The pushing device of claim 4, wherein:
the presser is elastically supported on the body portion by a spring member; and
the spring member is disposed between the upper step of the presser and the protruding member of the body portion.

6. The pushing device of claim 2, wherein a magnetic body is provided on a lower portion of the presser, and an electromagnet configured to selectively form an attractive force relative to the magnetic body is provided on the insertion groove portion.

7. The pushing device of claim 2, wherein at least one guide protrusion longitudinally formed in a vertical direction is provided along an outer circumference of the presser, and a guide groove portion configured to have the guide protrusion inserted thereinto and guide movement of the presser is formed in an inner wall of the body portion in which the through-hole is formed.

8. The pushing device of claim 1, wherein a magnetic body is installed on a contact portion between the presser and the press button to prevent sliding of the presser.

9. The pushing device of claim 1, further comprising:
a measurer configured to count a number of times an external force is applied to the presser and the presser presses the press button; and
a display configured to output the number of times the press button is pressed that is counted by the measurer.

10. The pushing device of claim 9, wherein the measurer measures click information of the press button provided on the drug delivery device to count the number of times the press button is pressed.

11. The pushing device of claim 3, further comprising:
a rotary gear portion mounted on the body portion and configured to generate a rotational force; and
an engaging portion attached to the support groove portion and engaged with the rotary gear portion to allow the presser to move in the longitudinal direction as the rotary gear portion rotates.

12. The pushing device of claim 3, further comprising:
a screw portion mounted on the body portion and configured to generate a rotational force; and
a screw coupling portion formed in the form of screw threads on the support groove portion and engaged with the screw portion to allow the presser to move in the longitudinal direction as the screw portion rotates.
